(19) United States
(12) Patent Application Publication
(10) Pub. No.: US 2023/0025743 A1
(43) Pub. Date: Jan. 26, 2023

(54) RUNTIME OPTIMISED ARTIFICIAL VISION

(71) Applicant: COMMONWEALTH SCIENTIFIC AND INDUSTRIAL RESEARCH ORGANISATION, Acton (AU)

(72) Inventors: Nariman HABILI, Acton (AU); Jeremy OORLOFF, Acton (AU); Nick BARNES, Acton (AU)

(21) Appl. No.: 17/782,304

(22) PCT Filed: Dec. 2, 2020
(86) PCT No.: PCT/AU2020/051308

§ 371 Date: Jun. 3, 2022

(30) Foreign Application Priority Data
Dec. 5, 2019 (AU) 2019904612

(51) Int. Cl. A61N 1/36; A61N 1/02; G06T 7/50; G06T 7/11; G06T 7/73; G06T 7/194

(52) U.S. Cl. CPC A61N 1/36046; A61N 1/025; G06T 7/50; G06T 7/11; G06T 7/73; G06T 7/194; A61N 1/0543

(57) ABSTRACT

A method for creating artificial vision with an implantable visual stimulation device. The method comprises receiving image data comprising, for each of multiple points of an image, a depth value, performing a local background enclosure calculation on the image data to determine salient object information, and generating a visual stimulus to visualise the salient object information using the implantable visual stimulation device. Performing the local background enclosure calculation is based on a subset of the multiple points of the input image, and the subset of the multiple points is defined based on the depth value of the multiple points.

### CROSS-REFERENCE TO RELATED APPLICATIONS

The present application claims priority from Australian Provisional Patent Application No 2019904612 filed on 5 Dec. 2019, the contents of which are incorporated herein by reference in their entirety.

### TECHNICAL FIELD

Aspects of this disclosure relate generally to the creation of artificial vision stimulus for use with an implantable visual stimulation device, and more specifically to systems and methods for optimising the efficacy of the same.

### BACKGROUND

Artificial vision systems which include implantable vision stimulation devices provide a means to provide vision information to a vision impaired user. An exemplary artificial vision system comprises an external data capture and processing component, and a visual prosthesis implanted in a vision impaired user, such that the visual prosthesis stimulates the user's visual cortex to produce artificial vision.

The external component includes an image processor, and a camera and other sensors configured to capture image of a field of view in front of a user. Other sensors may be configured to capture depth information, information relating to the field of view or information relating to the user. An image processor is configured to receive and convert this image information into electrical stimulation parameters, which are sent to a visual stimulation device implanted in the vision impaired user. The visual stimulation device has electrodes configured to stimulate the user's visual cortex, directly or indirectly, so that the user perceives an image comprised of flashes of light (phosphene phenomenon) which represent objects within the field of view.

A key component of visual interpretation is the ability to rapidly identify objects within a scene that stand out, or are salient, with respect to their surroundings. The resolution of the image provided to a vision impair user via an artificial vision system is often limited by the resolution and colour range which can be reproduced on the user's visual cortex by the stimulation probes. Accordingly, there is an emphasis on visually highlighting the objects, in the field of view, which appear to be salient to the user. Accordingly, it is important for an artificial vision system to accurately determine the location and form of salient objects, so that it may effectively present the saliency information to the user.

Due to the need for a wearable, lightweight prosthesis with a long lasting battery life, the processing power of the processing component of artificial vision systems are often limited. Furthermore, an artificial vision system may be required to provide object saliency information in a timely manner, to accommodate movement of the user or movement of the salient objects relative to the user. In such situations, it is beneficial to have a highly responsive solution for determining salient objects. This may also be referred to as “real-time”, which means within this disclosure, that a processor can perform the calculation within a frame rate that allows the user to continuously perceive a changing environment or changing viewing direction, such as 10, 20 or 40 frames/second or any other higher or lower frame rate.

Accordingly, there is a need to calculate and provide object saliency information, for artificial vision systems, at an optimised efficiency.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

A method for creating artificial vision with an implantable visual stimulation device is provided. The method comprises receiving image data comprising, for each of multiple points of an image, a depth value and one or more light intensity values; performing a local background enclosure calculation on the image data to determine salient object information; and generating a visual stimulus to visualise the salient object information using the implantable visual stimulation device, wherein performing the local background enclosure calculation is based on a subset of the multiple points of the input image, and wherein the subset of the multiple points is defined based on the depth value of the multiple points.

The method of claim 1 may further comprise spatially segmenting the image data into a plurality of superpixels, wherein each superpixel comprises one or more of the multiple points of the image, and wherein the subset of the multiple points comprises a subset of the plurality of superpixels.

The subset of superpixels may be defined based on a calculated superpixel depth value of the superpixels. Each of the superpixels in the subset of superpixels may have a superpixel depth value which is less than a predefined maximum object depth threshold. The calculated superpixel depth may be calculated as a function of the depth values of each of the one or more multiple points of the image that comprise the superpixel. The depth value of each of the multiple points in the subset of multiple points may be less than a predefined maximum depth threshold.

The subset of superpixels may be further defined based on a spatial location of the superpixel within the image, relative to the location of a phosphene location of a phosphene array. The selected superpixels may be collocated with the phosphene location.

Performing a local background enclosure calculation may comprise calculating a neighbourhood surface score based on the spatial variance of at least one superpixel within the image from one or more corresponding neighbourhood surface models, wherein the one or more neighbourhood surface models are representative of one or more corresponding regions neighbouring the superpixel.

The subset of superpixels may be further defined based on a spatial location of the superpixel within the image, relative to an object model information, which represents the location and form of predetermined objects within the image.

The method may further comprise adjusting the salient object information to include the object model information. The method may further comprise performing post-processing of the salient object information, wherein the post-processing comprises performing depth attenuation, saturation suppression and or flicker reduction.

A artificial vision device for creating artificial vision with an implantable visual stimulation device is provided. The artificial vision device comprises an image processor configured to receive image data comprising, for each of multiple points of an image, a depth value and one or more light intensity values; perform a local background enclosure calculation on the image data to determine salient object information; and generate a visual stimulus to visualise the salient object information using the implantable visual stimulation device, wherein performing the local background enclosure calculation is based on a subset of the multiple points of the input image and the subset of the multiple points is defined based on the depth value of the multiple points.

The artificial vision device may further comprise spatially segmenting the image data into a plurality of superpixels, wherein each superpixel comprises one or more of the multiple points of the image, and wherein the subset of the multiple points comprises a subset of the plurality of superpixels.

### BRIEF DESCRIPTION OF DRAWINGS

Examples will now be described with reference to the following drawings, in which:

FIG. 1 is block diagram illustrating an artificial vision system comprising an image processor in communication with a visual stimulation device;

FIG. 2 is a flowchart illustrating a method, as performed by an image processor, of generating visual stimulus;

FIG. 3 is a flowchart illustrating a method, as performed by an image processor, of receiving image data;

FIG. 4a illustrates a representation of a scene, and a magnified section of the same;

FIG. 4b-d illustrate the segmentation of the magnified section of FIG. 4a into a plurality of superpixels, and the selection of a subset of said superpixels;

FIG. 5 is a flowchart illustrating a method, as performed by an image processor, of calculating local background enclosure results.

### DESCRIPTION OF EMBODIMENTS

This disclosure relates to image data including a depth channel, such as from a laser range finder, ultrasound, radar, binocular/stereoscopic images or other sources of depth information.

An artificial vision device can determine the saliency of an object within a field of view represented by an image of a scene including a depth channel, by measuring the depth contrast between the object and its neighbours (i.e. local scale depth contrast) and the object and the rest of the image (i.e. global scale depth contrast).

Salient objects within a field of view tend to be characterised by being locally in front of surrounding regions, and the distance between an object and the background is not as important as the observation that the background surrounds the object for a large proportion of its boundary. The existence of background behind an object, over a large spread of angular directions around the object indicates pop-out structure of the object and thus implies high saliency of the object. Conversely, background regions in the field of view are less likely to exhibit pop-out structures, and may be considered to be less salient.

A technique for determining the saliency of an object in a field of view, based on these principles, is the calculation of a local background enclosure for candidate regions within an image of the field of view. Such a method has been described in “Local background enclosure for RGB-D salient object detection” (Feng D, Barnes N, You S, et al., Proceedings of the IEEE Conference on Computer Vision and Pattern Recognition, 2016, 2343-2350) [1], which is incorporated herein by reference.

The Local Background Enclosure (LBE) technique measures salientcy within an image based on depth information corresponding to pixels of the image. Specifically, the LBE technique analyses an object and more particularly, a candidate region that is a part of that object. A candidate region can be a single pixel or multiple pixels together in a regular or irregular shape.

The LBE technique defines a local neighbourhood around a candidate region and determines the spread and size of angular segments of pixels within that local neighbourhood (such as pixels within a predefined distance) that contain background, noting that the background is defined with respect to the candidate region. That is, a first object in front of a background plan may be part of the background of a second object that is in front of the first object.

The LBE technique applies a depth saliency feature that incorporates at least two components. The first, which is broadly proportional to saliency, is an angular density of background around the region. This encodes the intuition that a salient object is in front of most of its surroundings. The second feature component, which is broadly inversely proportional to saliency, is the size of the largest angular region containing only foreground, since a large value implies significant foreground structure surrounding the object.

The calculation of LBE to determine salient objects within an image can require significant computational capacity. For some applications, the image processing must be performed in a timely manner (i.e. real time) on a wearable processing device. However, the computational capacity required to perform LBE calculations may be prohibitively high. Furthermore, there is a need to calculate object saliency information promptly in order to provide the indicative visual stimulation to the user, especially in situations in which there is movement of the object or the user.

For at least these reasons, it is desirable to reduce the computational complexity of the LBE calculations. As will described in relation to the enclosed figures, the computational complexity of the LBE for an image may be reduced through the identification of select subsets of the image for which LBE calculations may be performed. LBE calculations for the remainder of the image may be forgone, thus reducing the computational complxity of the LBE calculations for the image.

The method by which image subsets are selected for LBE calculations will be described in relation to the following exemplary embodiments.

### Artificial Vision Device

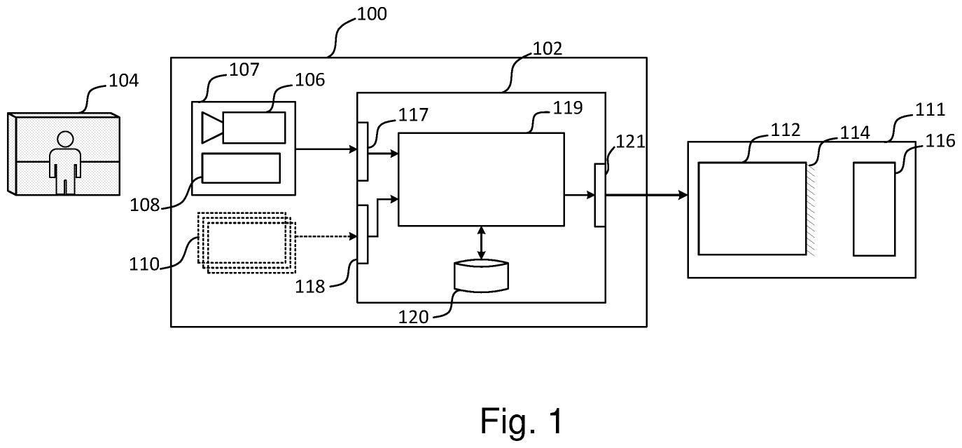

FIG. 1 is a block diagram illustrating an exemplary structure of an artificial vision device 100 which is configured to generate a visual stimulus, representative of a scene 104, for a vision impaired user 111. In particular, the artificial vision device 100 is configured to generate a representation of object saliency, for objects within the scene 104, for the vision impaired user. The scene 104 represents the physical environment of the user and is naturally three dimensional.

The vision impaired user 111 has an implanted visual stimulation device 112 which stimulates the user's visual cortex 116, either directly or indirectly, via electrodes 114 to produce artificial vision.

The artificial vision device may comprise a microprocessor based device, configured to be worn on the person of the user. The artificial vision device 100 illustrated in FIG. 1, includes an image sensor 106, a depth sensor 108 and an image processor 102. In other embodiments the image and depth sensors may be located external to the artificial vision device 100.

The aim is to enable the vision-impaired user to perceive salient objects within the view of the image sensor 106. In particular, the aim is to generate a stimulation signal, such that the user perceives salient objects as highlighted structures. For example, the user may, as a result of the stimulation, perceive salient objects as white image structures and background as black image structures or vice versa. This may be considered similar to ‘seeing’ a low resolution image. While the resolution is low, the aim is to enable the vision-impaired user to navigate everyday scenarios with the help of the disclosed artificial vision system by providing salient objects in sufficient detail and frame rate for that navigation and the avoidance of immediate dangers.

The image processor 102 receives input data representing multiple points (i.e. pixels) of the scene 104 from an image sensor 106, and a depth sensor 108. The image sensor 106 may be a high resolution digital camera which captures luminance information representing the field of view of the scene 104 from the camera's lens, to provide a two-dimensional pixel representation of the scene, with brightness values for each pixel. The image sensor 106 may be configured to provide the two-dimensional representation of the scene in the form of greyscale image or colour image.

The depth sensor 108 captures a representation of the distance of points in the scene 104 from the depth sensor. The depth sensor provides this depth representation in the form of a depth map which indicates a distance measurement for each pixel in the image. The depth map is created by computing stereo disparities between two space-separated parallel cameras. In one example, the depth sensor is a laser range finder that determines the distance of points in the scene 104 from the sensor by measuring the time of flight and multiplying the measured time of flight by the speed of light and divide by two to calculate a distance. In other examples, the pixels of the depth map represent the time of flight directly noting that a transformation that is identical for all pixels should not affect the disclosed method, which relies on relative differences in depth and not absolute values of the distance.

The image sensor 106 and the depth sensor 108 may be separate devices. Alternatively, they may be a single device 107, configured to provide the image and depth representations as separate representations, or to combine the image and depth representations into a combined representation, such as an RGB-D representation. An RGB-D representation is a combination of an RGB image and its corresponding depth image. A depth image is an image channel in which each pixel value represents the distance between the image plane and the corresponding point on surface within the RGB image. So, when reference is made herein to an ‘image’, this may refer to a depth map without RGB components since the depth map essentially provides a pixel value (i.e. distance) for each pixel location. In other words, bright pixels in the image represent close points of the scene and dark pixels in the image represent distant points of the scene (or vice versa).

For simplicity, the image sensor 106 and the depth sensor 108 will be described herein as a single device which is configured to capture an image in RGB-D. Other alternatives to image capture may, of course, also be used.

In other embodiments, the image processor 102 may receive additional input from one or more additional sensors 110. The additional sensors 110 may be configured to provide information regarding the scene 104, such as contextual information regarding salient objects within the scene 104 or categorisation information indicating the location of the scene 104. Alternatively or additionally, the sensors 110 may be configured to provide information regarding the scene 104 in relation to the user, such as motion and acceleration measurements. Sensors 110 may also include eye tracking sensors which provide an indication of where the user's visual attention is focused.

The image processor 102 processes input image and depth information and generates visual stimulus in the form of an output representation of the scene 104. The output representation is communicated to a visual stimulation device 112, implanted in the user 111, which stimulates the user's visual cortex 116 via electrodes 114.

The output representation of the scene 104 may take the form, for example, of an array of values which are configured to correspond with phosphenes to be generated by electrical stimulation of the visual pathway of a user, via electrodes 114 of the implanted visual stimulation device 112. The implanted visual stimulation device 112 drives the electrical stimulation of the electrodes in accordance with the output representation of the scene 104, as provided by the image processor 102.

The output data port 121 is connected to an implanted visual stimulation device 112 comprising stimulation electrodes 114 arranged as an electrode array. The stimulation electrodes stimulate the visual cortex 116 of a vision impaired user. Typically, the number of electrodes 114 is significantly lower than the number of pixels of camera 106. As a result, each stimulation electrode covers an area of the scene 104 captured by multiple pixels the sensors 107.

Typically, electrode arrays 114 are limited in their spatial resolution, such as 8×8, and in their dynamic range, that is, number of intensity values, such as 3 bit resulting in 8 different values; however, the image sensor 106 can capture high resolution image data, such as 640×480 with 8 bit.

Often, the image processor 102 is configured to be worn by the user. Accordingly, the image processor may be a low-power, battery-operated unit, having a relatively simple hardware architecture.

In an example, as illustrated in FIG. 1, the image processor 102 includes a microprocessor 119, which is in communication with the image sensor 106 and depth sensor 108 via input 117, and is communication with other sensors 110 via input 118. The microprocessor 119 is operatively associated with an output interface 121, via which image processor 102 can output the representation of the scene 104 to the visual stimulation device 112.

It is to be understood that any kind of data port may be used to receive data on input ports 117 and 118 and to send data on output port 121, such as a network connection, a memory interface, a pin of the chip package of processor 119, or logical ports, such as IP sockets or parameters of functions stored in memory 120 and executed by processor 119.

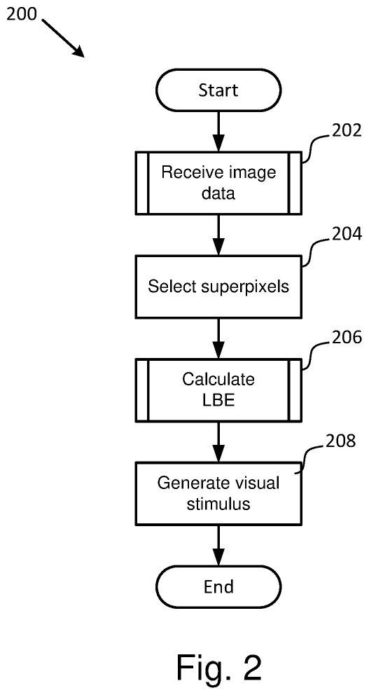

The microprocessor 119 is further associated with memory storage 120, which may take the form of random access memory, read only memory, and/or other forms of volatile and non-volatile storage forms. The memory 120 comprises, in use, a body of stored program instructions that are executable by the microprocessor 119, and are adapted such that the image processor 102 is configured to perform various processing functions, and to implement various algorithms, such as are described below, and particularly with reference to FIGS. 2 to 6.

The microprocessor 119 may receive data, such as image data, from memory storage 120 as well as from the input port 117. In one example, the microprocessor 119 receives and processes the images in real time. This means that the microprocessor 119 performs image processing to identify salient objects every time a new image is received from the sensors 107 and completes this calculation before the sensors 107 send the next image, such as the next frame of a video stream.

It is to be understood that, in other embodiments, the image processor 102 may be implemented via software executing a general-purpose computer, such as a laptop or desktop computer, or an application specific integrated device or a field programmable gate array. Accordingly the absence of additional hardware details in FIG. 1 should not be taken to indicate that other standard components may not be included within a practical embodiment of the invention.

### Method for Creating Artificial Vision

FIG. 2 illustrates a method 200 performed by the image processor 102, for creating artificial vision with an implantable visual stimulation device 112. Method 200 may be implemented in software stored in memory 120 and executed on microprocessor 119. Method 200 is configured through the setting of configuration parameters, which are stored in memory storage 120.

In step 202, the image processor 102 receives image data from the RGB_D camera 107. The image data comprises an RGB image, of dimensions x by y pixels, and a corresponding depth channel. In one example, the image data only comprises the depth channel.

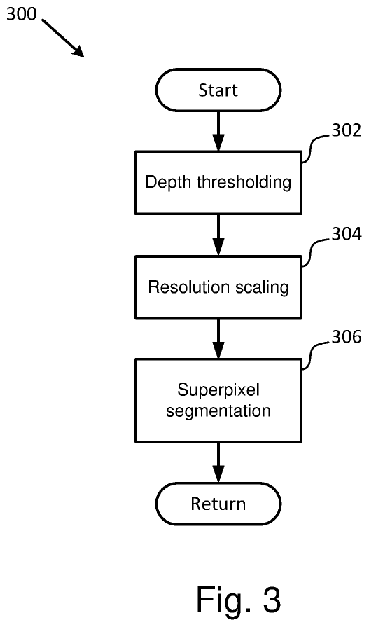

The image processor 102 pre-processes the received image data to prepare the data for subsequent processing. Method 300 in FIG. 3 illustrates the steps of pre-processing the received image data. In step 302, image processor 102 applies threshold masks to the depth image to ensure the pixels of the depth image are each within the defined acceptable depth range. The acceptable depth range for performing visual stimulation processing may be defined through configuration parameters which represent a maximum depth threshold and a minimum depth threshold. The depth threshold configuration parameters may vary in accordance with the type of scene being viewed, contextual information or the preferences of the user. The depth image may also be smoothed to reduce spatial or temporal noise. It is noted here that some or all configuration parameters may be adjusted either before the device is implanted or after implantation by a clinician, a technician or even the user itself to find the most preferable setting for the user.

In step 304, the image provided by image sensor 106 may be modified to reduce the spatial resolution of an image, and hence to reduce the number of pixels to be subsequently processed. The image may be scaled in the horizontal and vertical dimensions, in accordance with configuration parameters stored in the image processor.

In one example, image data of a reduced spatial resolution is determined by selecting every second pixel of the higher resolution image data. As a result, the reduced spatial resolution is half the high resolution. In other examples, other methods for resoution scaling may be applied.

In step 306, the image processor segments the RGB-D image, represented by pixel grid I(x, y). For computational efficiency and to reduce noise from the depth image, instead of directly working on pixels, the image processor segments the input RGB-D image into a set of superpixels according to their RGB value. In other examples, the image processor segments the input image data into a set of superpixels according to their depth values. This means, the input image does not necessarily have to include colour (RGB) or other visual components but could be purely a depth image. Other ways of segmentation may equally be used. In other words, image segmentation is the process of assigning a label (superpixel ID) to every pixel in an image such that pixels with the same label share certain characteristics (and belong to the same superpixel).

A superpixel is a group of spatially adjacent pixels which share a common characteristic (like pixel intensity, or depth). Superpixels can facilitate artificial vision algorithms because pixels belonging to a given superpixel share similar visual properties. Furthermore, superpixels provide a convenient and compact representation of images that can facilitate fast computation of computationally demanding problems.

### SLIC Superpixel Segmentation

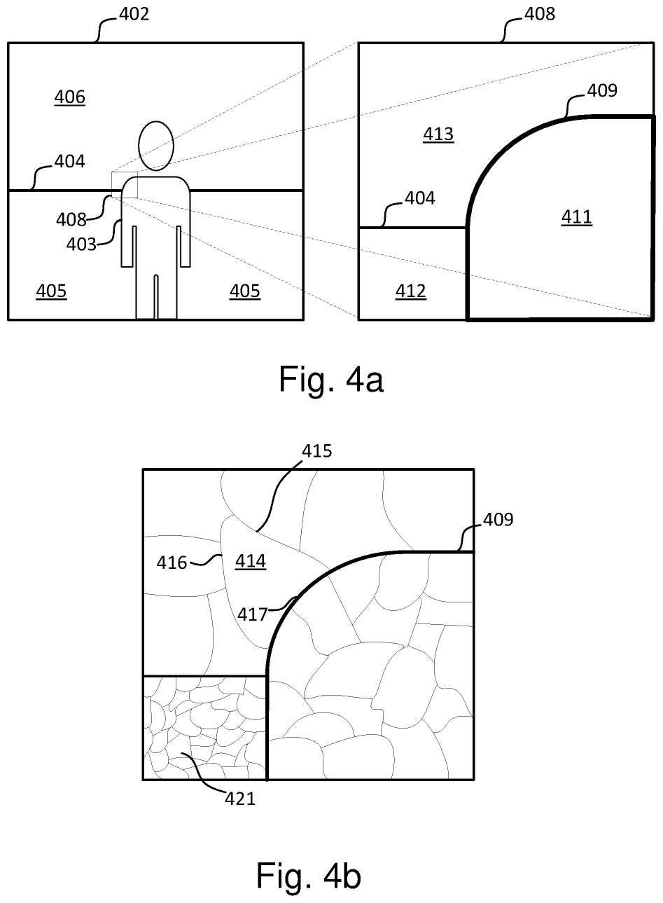

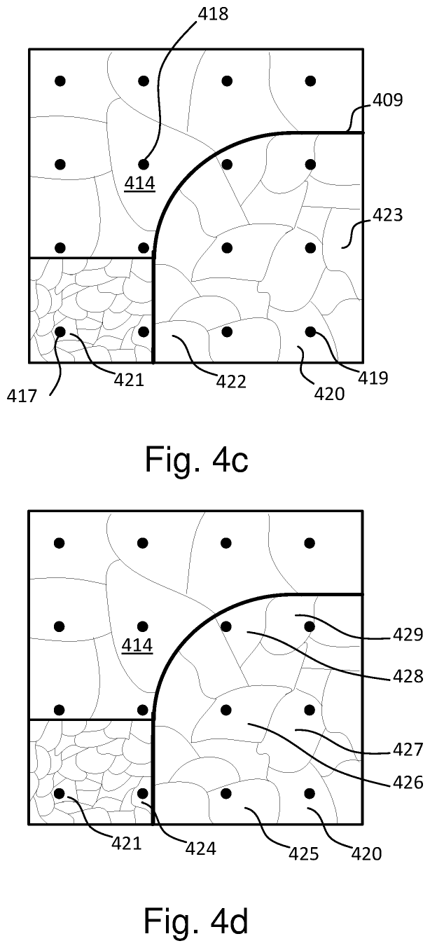

In the example of FIG. 4, the image processor 102 utilises the Simple Linear Iterative Clustering (SLIC) [2] algorithm to perform segmentation; however, it is noted that other segmentation algorithms may be applied. The SLIC segmentation algorithm may be applied through use of the OpenCV image processing library. The SLIC segmentation process is configured through the setting of configuration parameters, including a superpixel size parameter which determines the superpixel size of the returned segment, and a compactness parameter which determines the compactness of the superpixels within the image.

The processing power required to perform SLIC segmentation depends upon the resolution of the image, and the number of pixels to be processed by the segmentation algorithm. The resolution scaling step 304 assists with reducing the processing requirements of step 306 by reducing the number of pixels required to be processed by the segmentation algorithm.

### Segmentation Example

FIG. 4a illustrates a schematic image 402 of scene 104 captured by image sensor 106 and depth sensor 108. The image 402 is shown in monochrome, and omits natural texture, luminance and colour, for the purposes of explanation of the principles of the present disclosure.

The image 402 depicts a person 403 standing in front of a wall 405 which extends from the left hand side of the field of view to the right hand side of the field of view. The top 404 of the wall 405 is approximately at the shoulder height of the person 403. Behind the wall 405 is a void to a distant surface 406.

The depth sensor 108 has determined the depth of each of the pixels forming the image 402, and this depth information has been provided to the image processor 102. The depth information indicates that the person 403 is approximately 4 metres away from the depth sensor, the surface of the wall 405 is approximately 5 metres away from the depth sensor and the surface 406 is approximately 10 metres away from the depth sensor.

A magnified section 408 of the field of view representation 402 is provided in FIG. 4a. The magnified section 408 illustrates a section 411 of the shoulder of person 403, a section 412 of the wall 405, and a section 413 of the distant surface 406 behind the person's shoulder.

FIG. 4b illustrates a magnified section 408 of image 402, showing the result of performing the superpixel segmentation step 306 over the image 402. Specifically, FIG. 4b illustrates section 408 segmented into a plurality of superpixels. The superpixels each contain one or more adjacent pixels of the image. The superpixels are bounded by virtual segmentation lines. For example, superpixel 414, which includes pixels illustrating distance surface 406, is bounded by virtual segmentation lines 415, 416 and 417. Segmentation line 417 is collocated with the curve 409 of the person's shoulder 411.

It can be seen that the superpixels are of irregular shape and non-uniform size. In particular, superpixels representing the distant surface 406 are spatially larger, encompassing more pixels, than the superpixels representing the shoulder 411 of the person 403. Furthermore, the superpixels of the wall are spatially smaller and encompass few pixels. This is indicative of the wall having varying texture, luminance or chrominance.

Image processor 102 may use the superpixels determined in segmentation step 306 within Local Background Enclosure calculations to identify the presence and form of salient objects; however, performing an LBE calculation for each superpixel in the image requires a significant amount of processing power and time. Accordingly, following the segmentation step 306, the image processor 102 performs superpixel selection in step 204, prior to performing the LBE calculations in step 206.

Advantageously, in performing the select superpixels step 204, the image processor identifies a subset of all superpixel of the image 402 as selected superpixels for which a local background enclosure (LBE) is to be calculated. Thus, the image processor does not need to perform LBE calculations for all superpixels determined in segmentation step 306, and the computational complexity of the LBE calculations is therefore reduced.

### Superpixels Selection

In step 204, the image processor considers each phosphene location in an array of phosphene locations to determine which superpixel each phosphene location corresponds to, and whether the depth of the corresponding superpixel is within a configured object depth threshold.

The object depth threshold indicates the distance from the depth sensor 108 at which an object may be considered to be salient by the image processor. The object depth threshold may comprise a maximum object depth threshold and a minimum object depth threshold. The maximum distance at which an object would be considered not salient may depends upon on the context of the 3D spatial field being viewed by the sensors. For example, if the field of view is an interior room, objects that are over 5 meters away may not be considered to be salient objects to the user. In contrast, if the field of view is outdoors, the maximum depth at which objects may be considered salient may be significantly further.

If the depth of the superpixel corresponding with a phosphene location is not within the defined object depth threshold, the superpixel is not selected by the image processor for subsequent LBE calculation.

In FIG. 4c, a section of a phosphene array is shown overlaid over the magnified representation 408 of a section of the field of view. The section of phosphene array is depicted as a four by four array of dots. Each dot represents the approximate relative spatial location of an electrode which has been implanted into vision impaired user.

Each phosphene location depicted in FIG. 4c is collocated with a superpixel. For example, phosphene location 418 is collocated with superpixel 414, which depicts a section of the distant surface 406. Phosphene location 419 is collocated with superpixel 420, which depicts a section of the person's shoulder 411. Phosphene location 417 is collocated with superpixel 421 representing a section the wall 405. Notably, some superpixels, such as 422 and 423, are not collocated with a phosphene location, and such superpixels will not be selected by the image processor for subsequent LBE calculations.

The image processor may be configured to select two or more neighbouring superpixels for subsequent LBE calculations, in the event that a phosphene location is close to the boundary of two or more superpixels. The image processor may also be configured to detect when two or more phosphene locations are collocated with a single superpixel. In this case, the image processor may ensure that the superpixel is not duplicated within the list of selected superpixels.

For each superpixel that is collocated with a phosphene location, the image processor calculates a superpixel depth. A superpixel depth is a depth value that is representative of the depths of each pixel within the superpixel. The calculation method to determine the superpixel depth may be configured depending upon the resolution of the image, resolution of the depth image, context of the image or other configuration parameters. In the example of FIGS. 4a-d, a depth measurement is available for each pixel of the image, and image processor 102 calculates the superpixel depth via a non-weighted average of the depth measurements of all pixels within the superpixel. In another example, the image processor 102 calculates the superpixel depth via a weighted average of the depth measurements of all pixels, giving a larger weight to pixels located in a centre region of the superpixel. In yet another example, the superpixel depth may be a statistical mean depth value of the encompassed pixel depth values. It is to be understood that other methods of calculating the superpixel depth based upon the depths of the encompassed pixels may be used within the context of the artificial vision device and method as described herein.

In the example of FIG. 4a-d, superpixel 414 has a superpixel depth of 10 metres, superpixel 420 has a superpixel depth of 4 metres, and superpixel 421 has a superpixel depth of 5 metres.

In the example shown in FIGS. 4a-d, method 200 has been configured with a maximum object depth threshold configuration parameter of 7 metres, meaning that the system has been configured to not provide visual representations to the user of objects which are measured to be 7 or more metres away from the depth sensor 108.

The image processor 102 then selects superpixels which are collocated with a phosphene location, and have a superpixel depth which is less than the maximum object depth threshold configuration parameter.

FIG. 4d illustrates which superpixels are included in the list of selected superpixels, for this exemplary embodiment. Superpixels 420, 421, 424, 425, 426, 427, 428 and 429 are included in the list of selected superpixels. Notably, no superpixel which includes pixels representing the distant surface 406 is included in the list of selected superpixels, because the depth of the distant surface 406 exceeds the depth threshold configuration parameter. For example, superpixel 414 is not included in the list of selected superpixels.

The list of selected superpixels determined in step 204 is stored in memory for use in subsequent steps of method 300.

As described with reference to FIGS. 4a-d, the image processor may select a subset of the superpixels based upon a maximum object depth threshold. In another embodiment, the image processor may, in addition, or alternatively, select a subset of the superpixels based on a minimum object depth threshold. The application of a minimum object depth threshold may be of particular use in situations in which there is no need to detect the presence and form of salient objects that are within a certain close range to the depth sensor 108. An exemplary situation is when the field of view includes a salient object at close range for which the user is already aware, or for which the image processor has previously identified as being present. Accordingly, there may only be a need to detect additional salient objects at a mid-range depth. In this exemplary situation, the image processor appends the representation of the close range salient object to the visual stimulus after LBE calculations have been performed, thus reducing the number of LBE calculations that are required to be performed for a particular image frame. In another example, the close range salient object may be not represented in the visual stimulus generated by the image processor.

In yet another example, the image processor has access to an object model for the field of view 104, which comprises information representing the location and form of one or more predetermined objects within the field of view. The location and form of the predetermined objects may be determined by the image processor. Alternatively, the object model may be provided to the image processor. In this example, the image processor appends the object model information, which represents the location and form of one or more predetermined objects, to the salient object information after LBE calculations have been performed, thus reducing the number of LBE calculations that are performed for a particular image frame.

For some embodiments, or some situations, it may be desirable or feasible to calculate the LBE for every superpixel within the image. In this case, a configuration parameter may be set to indicate that the superpixel selection process may be omitted, and the output of step 204 will be a list of every superpixel in the image.

### Calculate LBE

In step 206, the image processor 102 calculates the local background enclosure (LBE) for each of the superpixels in the list of selected superpixels provided by step 204.

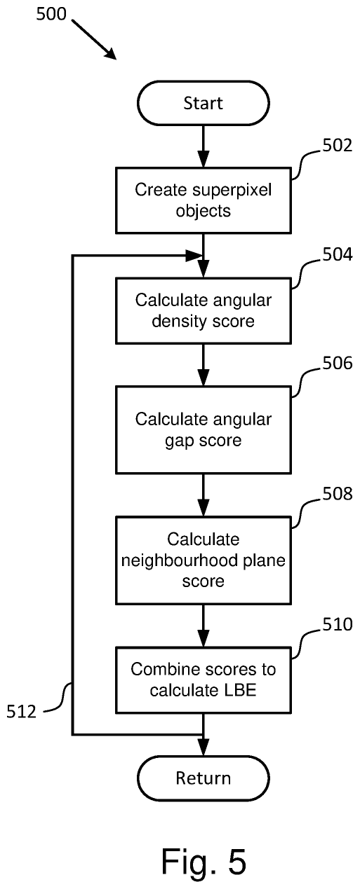

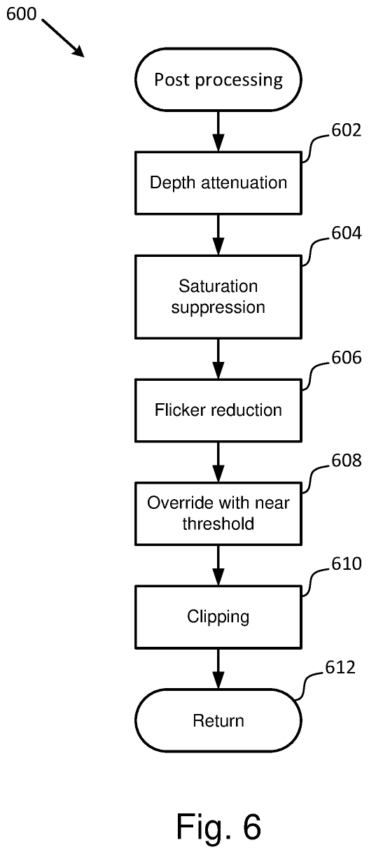

FIG. 5 illustrates the steps taken by the image processor 102 to calculate the LBE for the list of selected superpixels. In step 502, the image processor 102 creates superpixel objects for each superpixel in the list of selected superpixels by calculating the centroid of each superpixel, and the average depth of the pixels in each superpixel. When calculating the average depth, the method may ignore depth values equal to zero.

For each of the selected superpixels, the image processor additionally calculates the standard deviation of the depth, and a superpixel neighbourhood comprised of superpixels that are within a defined radius of the superpixel.

In steps 504 to 508, for each selected superpixel P, the image processor 102 calculates, based on superpixel's neighbourhood, an angular density score F, an angular gap score G, and, optionally, a neighbourhood surface score for each superpixel. These scores are combined to produce the LBE result S, for the superpixel.

### Angular Density Score

In step 504, the image processor calculates the angular density of the regions surrounding P with greater depth than P, referred to as the local background. A local neighbourhood N_{P} of P, consisting of all superpixels within radius r of P. That is, N_{P}={Q|Pc_{P}−c_{Q} P₂<r}, where c_{P} and c_{Q} are superpixel centroids.

The local background B(P,t) of P is defined as the union of all superpixels within a neighbourhood N_{P} that have a mean depth above a threshold t from P.

B(P,t)=∪{P′∈N_{P}|D(P′)>D(P)+t}, (1)

where D(P) denotes the mean depth of pixels in P.

Method 500 defines a function ƒ (P,B(P,t)) that computes the normalised ratio of the degree to which B(P,t) encloses P.

$$f ( P , B ( P , t ) ) = \frac{1}{2 π} {\int }_{0}^{2 π} I ( θ , P , B ( P , t ) ) d θ , \qquad ( 2 )$$

where I (θ,P,B(P,t))) is an indicator function that equals 1 if the line passing through the centroid of superpixel P with angle θ intersects B (P,t), and 0 otherwise.

Thus ƒ (P,B(P,t)) computes the angular density of the background directions. Note that the threshold t for background is an undetermined function. In order to address this, as frequently used in probability theory, we employ the distribution function, denoted as F(P). instead of the density function ƒ, to give a more robust measure. We define F(P) as:

F(P)=∫₀^{σ}ƒ(P,B(P,t))dt, (3)

where σ is the standard deviation of the mean superpixel depths within the local neighbourhood of P.

This is given by

$${σ}^{2} = \frac{1}{\vert B ( P , 0 ) \vert} {\sum }_{Q ∈ B ( P , 0 )} {( D ( Q ) - \overline{D} )}^{2} ,$$

where

$$\overline{D} = \frac{1}{\vert B ( P , 0 ) \vert} {\sum }_{Q ∈ B ( P , 0 )} D ( Q ) .$$

This implicitly incorporates information about the distribution of depth differences between P and its local background.

### Angular Gap Score

In step 506, the image processor 102 calculates an angular gap score G(P). The angular gap score provides an adjustment in the situation where two superpixels have similar angular densities; however, one of the two superpixels appears to have higher saliency due to background directions which are more spread out. To provide this adjustment, the method 500 applies the function g (P, Q) to find the largest angular gap of Q around P and incorporate this into the saliency score.

$$g ( P , Q ) = \frac{1}{2 π} \cdot {max}_{( {θ}_{1} , {θ}_{2} ) ∈ Θ} \{ \vert {θ}_{1} - {θ}_{2} \vert \} , \qquad ( 4 )$$

where Θ denotes the set of boundaries (θ₁, θ₂) of angular regions that do not contain background:

Θ={(θ₁,θ₂)|I(θ,P,Q)=0∀θ∈[θ₁,θ₂]}. (5)

The angular gap statistic is defined as the distribution function of 1−g:

G(P)=∫₀^{σ}1−g(P,B(P,t)) dt. (6)

The final Local Background Enclosure value is given by:

S(P)=F(P)·G(P). (7)

### Neighbourhood Surface Score

Optionally, the image processor 102 may be configured to calculate a third score, namely, a neighbourhood surface score which provides an adjustment to the LBE result to visually distinguish salient object which are located on or near a salient surface. For some surfaces within the scene, the angular density score and angular gap score provide a high LBE result, indicating that the surface is salient. As a result, superpixels which are located on the surface will be represented as highly salient regions, and visually highlighted in the visual stimulus provided to the user.

If an object is located in front of or on the salient surface, the visual highlighting of the surface may result in the foreground object not being visually distinguished, to the user, from the mid-ground salient surface. Accordingly, the neighbourhood surface score partially or fully suppresses the LBE result for a superpixel in cases where the superpixel lies on or close to a surface model.

To calculate the neighbourhood surface score, the image processor 102 obtains a neighbourhood surface model which is representative of a virtual surface in the neighbourhood of the superpixel. In one example, the neighbourhood surface model is calculated by the image processor 102. In another example, the neighbourhood surface model is provided to the image processor 102. In one example, the neighbourhood surface model is calculated as a best-fit model based on the spatial locations represents by pixels in a defined region neighbouring a superpixel.

The neighbourhood surface score for a superpixel is based on spatial variance of the superpixel from the neighbourhood surface model. In one example, the neighbourhood surface score for a superpixel is based on the number of pixels within the superpixel that are considered to be outliers from the neighbouring surface model. In another example, the neighbourhood surface score is based on the sum total of distances of the pixels within the superpixel from the neighbouring surface model.

If there is a high degree of spatial variance of a superpixel from the neighbourhood surface model, the image processor provides a high neighbourhood surface score, i.e. close to 1, as it is desirable to preserve the LBE result for objects that are not on the surface. If there is a low degree of spatial variance of a superpixel from the neighbourhood surface model, the region surrounding the superpixel is considered to be aligned with the surface and it is desirable to suppress the LBE result for this case by providing a low neighbourhood surface score, i.e. close to 0.

An exemplary neighbourhood surface score is 1.0−(1.0/(number of outliers—LBE_SURFACE_OUTLIER_THRESHOLD)*4); where LBE_SURFACE_OUTLIER_THRESHOLD is defaulted to 5. This function provides a neighbourhood surface score for a superpixel of 0 below LBE_SURFACE_OUTLIER_THRESHOLD and curves up to 1 sharply after.

The equation can be generalised to

$$S ( P ) = 1 - ( \frac{1}{{o}_{P} - T} * α )$$

$$where o = outliers , T = outlier threshold , and$$

$$α = a scaling co - efficient .$$

### Combining the Scores

In step 510, the image processor 102 combines the angular density score, the angular gap score and, optionally, the neighbourhood surface score to give the LBE value for a superpixel. In one embodiment, the scores are combined through an unweighted multiplication. In other embodiments, weighted or conditional multiplication methods may be used to combine the scores to produce an LBE value for a superpixel.

The image processor repeats 512 steps 504 to 510 for each superpixel in the list of selected superpixels as provided in step 204, in order to determine an LBE value for each of the selected superpixels. Notably, due to the superpixel selection step 204, phosphene locations which correspond with superpixels that have a superpixel depth outside the object depth threshold will not have a corresponding calculated LBE value.

### Determine Phosphene Values

Following the determination of the LBE value for each of the selected superpixels, the image processor 102 determines a phosphene value for each of the phosphene locations in the array of phosphene locations. For phosphene locations that are collocated with one of the selected superpixels, the phosphene value is determined to be the LBE value of that superpixel. For phosphene locations that are collocated with a non-selected superpixels, the phosphene value is determined to be zero.

The array of phosphene values represent salient object information regarding the field of view captured by the image and depth sensors. This salient object information may be visualised, by the visual stimulation device user, as an array of light intensities representing the form and location of salient objects.

### Post-Processing

Optionally, and depending upon the requirements and operational parameters of an embodiment, the image processor may perform post-processing on the array of phosphene values to improve the effectiveness of the salient object information.

An exemplary embodiment of the post-processing method is illustrated in FIG. 6. It is to be understood that method 600 is a non-limiting example of post-processing steps that an image processor may perform following the determination of the phosphene value for phosphene locations. In some embodiments, the image processor 102 may perform all of steps 602 to 612, in the order shown in FIG. 6. Alternatively, the image processor may perform only a subset of steps 602 to 612 and/or perform the steps of method 600 in an alternative order to that shown FIG. 6.

### Perform Depth Attenuation

In step 602, the image processor 102 may dampen each phosphene value in accordance with a depth attenuation configuration parameter. For example, the method may calculate a scaling factor according to: scale=1−(current_phosphene_depth* (1−depth_attenuation_percent))/max_distance which is then applied to the current phosphene value or

$$depthScale ( p ) = 1 - \frac{{d}_{p} - δ}{{d}_{max}}$$

$$where d = depth , δ = attenuation percentage .$$

The depth attenuation adjustment results in nearer objects being brighter and farther objects being dimmer. For example, if the depth_attenuation_percent is set to 50%, and max_distance was 4.0 m, a phosphene value that was representing distance of 4.0 m would be dimmed by 50%, and one at 2.0 m would be dimmed by 25%.

### Perform Saturation Suppression

In step 604, the image processor may perform saturation suppression by calculating the global phosphene saturation by taking the mean value of all phosphene values. If the mean value is greater than a defined saturation threshold configuration parameter, the image processor performs a normalisation on the image to reduce the value of some phosphene values and thereby remove some saturation. Removing saturation of the phosphene values has the effect of drawing out detail within the visual stimulus.

### Flicker Reduction

The image processor may also be configured to perform the step of flicker reduction 606. Flicker reduction is a temporal feature to improve image stability and mitigate noise from both the depth camera data and LBE. A flicker delta configuration parameter constrains the maximum amount a phosphene value can differ from one frame to the next, this is implemented simply by looking to the data from the last frame and making sure phosphene values do not change by more than this amount. Flicker reduction aims to mitigate flashing noise and enhances smooth changes in phosphene brightness.

Additionally, the image processor may be configured to set a phosphene value to 1 in the situation that a phosphene's depth value is closer than minimum depth. Furthermore, the image processor may be configured to clip or adjust phosphene values to accommodate input parameter restrictions of the implanted visual stimulation device.

### Generate Visual Stimulus

Once the image processor has calculated the LBE results for each of the selected superpixels, determined the phosphene value for each of the phosphene locations and has performed any post-processing functions configured for a specific embodiment, the image processor generates the visual stimulus. The image processor then communicates the visual stimulus to the visual stimulation device 112, via output 121.

The visual stimulus may be in the form of a list phosphene values, with one phosphene value for each phosphene location on the grid of phosphene locations. In another example, the visual stimulus may comprise differential values, indicating the difference in value for each phosphene location, compared to the corresponding phosphene value at the previous image frame. In other examples, the visual stimulus is a signal for each electrode and may include an intensity for each electrode, such as stimulation current, or may comprise the actual stimulation pulses, where the pulse width defines the stimulation intensity.

In one example, the phosphene locations correspond with the spatially arranged implanted electrodes 114, such that the low resolution image formed by the grid of phosphenes may be reproduced as real phosphenes within the visual cortex of the user. Real phosphenes is the name given to the perceptual artefact caused by electrical stimulation on an electrically stimulating visual prosthetic.

In one example, the simulated phosphene display consists of a 35×30 rectangular grid scaled to image size. Each phosphene has a circular Gaussian profile whose centre value and standard deviation is modulated by brightness at that point. In addition, phosphenes sum their values when they overlap. In one example, phosphene rendering is performed at 8 bits of dynamic range per phosphene, which is an idealised representation. In a different example, it is assumed that maximum neuronal discrimination of electrical stimulation is closer to a 3 bit rendering. In another example, there are different numbers of bits of representation at each phosphene, and this may change over time.

In response to receiving the visual stimulus output from the image processor 102, the implanted visual stimulation device 112 stimulates the retina via the electrodes 114 at intensities corresponding with the phosphene values provided for each electrode. The electrodes 114 stimulate the visual cortex of the vision impaired user 111, triggering the generation of real phosphene artefacts at intensities broadly corresponding with the phosphene values. These real phosphenes provide the user with artificial vision of salient objects with the field of view 104 of the sensors 107.

In one example, the method 200, and associated sub-methods 300, 500 and 600, are applied to frames of video data, and the image processor generates visual stimulus on a per frame basis, to be applied to the electrodes periodically.

In one example, the visual stimulus is further adjusted to suit the needs of a particular vision impaired user or the characteristics of the vision impairment of that user. Furthermore, the adjustment of the visual stimulus may change over time due to factors such as polarisation of neurons.

In one example, the image processor adapts to the perception of the user on a frame by frame basis, where the visual stimulus is adjusted based on aspects of the user, such as the direction of gaze of the user's eyes.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### REFERENCES

[1] Local background enclosure for RGB-D salient object detection, Feng D, Barnes N, You S, et al., Proceedings of the IEEE Conference on Computer Vision and Pattern Recognition, 2016, 2343-2350.

[2] SLIC superpixels compared to state-of-the-art superpixel methods, Achanta R, Shaji A, Smith K, et al., PAMI, 34(11):2274-2282, 2012.

1. A method for creating artificial vision with an implantable visual stimulation device, the method comprising:
receiving image data comprising, for each of multiple points of an image, a depth value;
performing a local background enclosure calculation on the image data to determine salient object information; and
generating a visual stimulus to visualise the salient object information using the implantable visual stimulation device,
wherein performing the local background enclosure calculation is based on a subset of the multiple points of the input image, and
wherein the subset of the multiple points is defined based on the depth value of the multiple points.

2. The method of claim 1, further comprising spatially segmenting the image data into a plurality of superpixels,
wherein each superpixel comprises one or more of the multiple points of the image, and
wherein the subset of the multiple points comprises a subset of the plurality of superpixels.

3. The method of claim 2, where the subset of the plurality of superpixels is defined based on a calculated superpixel depth value of the superpixels.

4. The method of claim 2, wherein each of the superpixels in the subset of the plurality of superpixels has a superpixel depth value which is less than a predefined maximum object depth threshold.

5. The method of claim 3, where the calculated superpixel depth is calculated as a function of the depth values of each of the one or more multiple points of the image that comprise the superpixel.

6. The method of claim 1, wherein the depth value of each of the multiple points in the subset of the multiple points is less than a predefined maximum depth threshold.

7. The method of claim 2, wherein the subset of the plurality of superpixels is further defined based on a spatial location of the superpixel within the image, relative to the location of a phosphene location of a phosphene array.

8. The method of claim 2, wherein the selected superpixels are collocated with the phosphene location.

9. The method of claim 1, wherein performing a local background enclosure calculation comprises calculating a neighbourhood surface score based on the spatial variance of at least one superpixel within the image from one or more corresponding neighbourhood surface models, wherein the one or more neighbourhood surface models are representative of one or more corresponding regions neighbouring the superpixel.

10. The method of claim 2, wherein the subset of the plurality of superpixels is further defined based on a spatial location of the superpixel within the image, relative to an object model information, which represents the location and form of predetermined objects within the image.

11. The method of claim 10, further comprising adjusting the salient object information to include the object model information.

12. The method of claim 1, further comprising performing post-processing of the salient object information, wherein the post-processing comprises performing depth attenuation, saturation suppression and or flicker reduction.

13. An artificial vision device for creating artificial vision with an implantable visual stimulation device, the artificial vision device comprising an image processor configured to:
receive image data comprising, for each of multiple points of an image, a depth value;
perform a local background enclosure calculation on the image data to determine salient object information; and
generate a visual stimulus to visualise the salient object information using the implantable visual stimulation device,
wherein performing the local background enclosure calculation is based on a subset of the multiple points of the input image and the subset of the multiple points is defined based on the depth value of the multiple points.

14. The artificial vision device of claim 13, further comprising spatially segmenting the image data into a plurality of superpixels,
wherein each superpixel comprises one or more of the multiple points of the image, and
wherein the subset of the multiple points comprises a subset of the plurality of superpixels.

15. The artificial vision device of claim 14, where the subset of the plurality of superpixels is defined based on a calculated superpixel depth value of the superpixels.

16. The artificial vision device of claim 15, wherein each of the superpixels in the subset of the plurality of superpixels has a superpixel depth value which is less than a predefined maximum object depth threshold.

17. The artificial vision device of claim 15, where the calculated superpixel depth is calculated as a function of the depth values of each of the one or more multiple points of the image that comprise the superpixel.

18. The artificial vision device of claim 13, wherein the depth value of each of the multiple points in the subset of multiple points is less than a predefined maximum object depth threshold.

19. The artificial vision device of claim 14, wherein the subset of the plurality of superpixels is further defined based on a spatial location of the superpixel within the image, relative to the location of a phosphene location of a phosphene array.

20. The artificial vision device of claim 14, wherein the selected superpixels are collocated with the phosphene location.
